(19) United States
(12) Patent Application Publication
(10) Pub. No.: US 2014/0200486 A1
(43) Pub. Date: Jul. 17, 2014

(54) SYSTEM AND METHOD FOR CONTINUOUS MONITORING OF A HUMAN FOOT FOR SIGNS OF ULCER DEVELOPMENT

(71) Applicant: QUAERIMUS, INC., Newark, CA (US)

(72) Inventors: KATE LEEANN BECHTEL, PLEASANT HILL, CA (US); RICHARD W. O'CONNOR, REDWOOD CITY, CA (US); DAVID EDWARD GOODMAN, GREENBRAE, CA (US); JIVKO M. MIHAYLOV, SAN JOSE, CA (US)

(73) Assignee: QUAERIMUS, INC., NEWARK, CA (US)

(21) Appl. No.: 13/744,154

(22) Filed: Jan. 17, 2013

(51) Int. Cl. A61B 5/103

(52) U.S. Cl. CPC A61B 5/1036
USPC 600/592

(57) ABSTRACT

The present invention pertains to a system and method for monitoring a human foot by measuring pressures applied to regions of the foot or by measuring another tissue-health related condition. A light source in the 400 nm to 1400 nm range and a detector can be embedded in a wearable article that contacts tissue while in use, spaced 200 μm to 1 cm apart, and measure a tissue hemoglobin condition. A pressure-sensing array may be read by a low-power control circuit, and a power source can be incorporated in the article. An external processing unit wirelessly coupled to the control circuit can relate pressures measured with counts that are associated with injury risk, and an alert system can notify a patient if the counts exceed a predetermined threshold. A relationship between pressure experienced by a region of tissue and the risk of ulcer development in that region may be derived.

### FIELD OF THE INVENTION

The present invention pertains to technology for diagnosing and preventing pressure-induced tissue injuries. The present invention pertains more particularly to technology for diagnosing and preventing Diabetic Foot Ulcers.

### BACKGROUND

Patients that suffer from diabetic neuropathy can gradually lose sensing function in their extremities, particularly their feet. Yet neuropathic patients can maintain motor function, such that they can continue walking on, e.g. applying pressure and exposing to possible injury, feet for which they may have lost nociception. Nociception is the sensory or neural capacity to recognize adverse or noxious stimuli. With loss of nociception, patients can have a vastly increased risk of developing a serious injury or ulcer on their feet; when a patient does not feel a pressure point or wound as painful or uncomfortable, he or she may not notice an issue before it has progressed to a serious, highly noticeable degree. For example, Diabetic Foot Ulcers (DFU's) are sometimes only recognized when blood begins to appear on a patient's sock, a point at which ischemia, e.g. tissue death that may have started at an internal tissue region, has already progressed through tissue to an outer layer, and amputation may be necessary. Some studies have shown that 15% to 25% of diabetic patients are likely to develop a Diabetic Foot Ulcer (DFU) in their lifetimes. DFU's can lead to hospitalization, amputation, and ultimately a heightened patient morbidity risk.

There are presently no solutions that function as an effective nociception replacement to prevent neuropathic patients from developing DFU's or similar injuries. Most existing pressure-analysis systems are very expensive, intended for use by shoe manufacturers to evaluate the load profile of a test subject or a shoe under development, laboratories, coaches or physical therapists for single-session evaluation, and so forth. Furthermore, absolute values of pressure applied to tissue may not be enough to predict ulcer formation in a given patient, given the many patient-specific that may affect ulcer formation. What is needed is a system that can provide auxiliary nociceptive perception and can detect warning signs of early-stage ulcer development.

### SUMMARY

The present invention pertains to apparatus for monitoring a human foot by measuring pressures applied to regions of the foot with an array of pressure sensors or by measuring another tissue-health related condition, such as nonblanchable erythema or oxygen saturation, with a light source and detector. The light source and detector can be embedded in a wearable article that contacts tissue while in use, such as a sock, slipper, or patch, and can be spaced between 200 nm and 1 cm from one another. The source and sensor can be embedded at points configured to contact a load-bearing region of the tissue. The light source may emit light of one or two wavelengths between 400 nm and 1400 nm, or further between 800 nm and 820 nm, and may have a diameter less than 1 mm.

The array of pressure sensors may be read and controlled by a low-power control circuit, such that a power source incorporated in the article can power the array and the control circuit. An external processing unit wirelessly coupled to the control circuit can relate pressures measured with counts that are associated with injury risk, and an alert system can notify a patient if the counts exceed a predetermined threshold.

The present invention also pertains to a method for determining a relationship between an amount of pressure experienced by a region of tissue and the risk of ulcer development in that region by measuring both the pressure experienced by that region and a tissue hemoglobin condition, such as total hemoglobin or the ratio of oxyhemoglobin to deoxyhemoglobin, in the region over a predetermined period. These measurements can be acquired by wearable articles configured to measure pressure and the tissue hemoglobin condition. The tissue hemoglobin condition can be analyzed for signs indicative of ulcer development. This method can further determine a pressure value that affects the tissue hemoglobin condition and use that value as a threshold value triggering an alert during future monitoring of that region. This threshold or other information can be transmitted to an electronic medical record of the patient.

These and other objects and advantages of the various embodiments of the present invention will be recognized by those of ordinary skill in the art after reading the following detailed description of the embodiments that are illustrated in the various drawing figures.

### BRIEF DESCRIPTION OF THE DRAWINGS

The present invention is illustrated by way of example, and not by way of limitation, in the figures of the accompanying drawings and in which like reference numerals refer to similar elements.

FIG. 1 is a diagram showing an exemplary pressure-sensing insole of one embodiment of the present invention.

FIG. 2 is a diagram showing a cross-section of one embodiment comprising an array of capacitive sensors.

FIG. 3 is a diagram showing an exemplary processing circuit of one embodiment of the present invention.

FIG. 4 is a diagram showing an insole sensor configuration of one embodiment of the present invention for monitoring statistically likely sites of ulcer development.

FIG. 5 is a flow diagram representing a method of data-binning of one embodiment of the present invention including load consideration.

### DETAILED DESCRIPTION

Reference will now be made in detail to embodiments of the present invention, examples of which are illustrated in the accompanying drawings. While the invention will be described in conjunction with these embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of embodiments of the present invention, numerous specific details are set forth in order to provide a thorough understanding of the present invention. However, it will be recognized by one of ordinary skill in the art that the present invention may be practiced without these specific details. In other instances, well-known methods, procedures, components, and circuits have not been described in detail as not to unnecessarily obscure aspects of the embodiments of the present invention.

Embodiments of the present invention may comprise continuous-monitoring devices, periodic-monitoring devices, data transmitters and receivers, processors, displays, or any subset or combination thereof that in conjunction with one another may serve as a system for monitoring neuropathic patients for potential injury or ulceration. These systems may monitor specific tissue regions, collect data for analysis by medical practitioners, alert patients of conditions likely to cause ulceration, or serve other preventative and diagnostic functions.

A continuous-monitoring device may be a device worn by a patient on or near an extremity affected by neuropathy. A continuous-monitoring device may be low-profile, lightweight, convenient, and comfortable for patient wear during day-to-day activity. A continuous-monitoring device may continuously, e.g. at predetermined intervals that are short relative to an intended time-interval of device wear, collect data on one or more conditions affecting tissue or extremity health. A continuous-monitoring device may also or alternatively collect data when triggered based on another system input.

A periodic-monitoring device may be a device tailored to in-home or medical office use for examining tissue or extremity health monthly, weekly, daily, or more frequently. A periodic-monitoring device may measure the same parameter or parameters as a continuous-monitoring device or may measure alternative parameters relevant to tissue or extremity health. In one embodiment of the present invention, a periodic-monitoring device can comprise an image capture system configured to image the bottom of a patient's foot or feet. The bottoms or soles of a patient's feet can be particularly difficult to see without assistance, increasing the likelihood of an undetected injury or site of ulcer development. The bottoms or soles of a patient's feet can also be particularly susceptible to injury or ulceration from the pressure loads applied during walking, standing, and other activity. In other embodiments of the present invention, a periodic monitoring device can be configured for hyperspectral imaging, electrical impedance tomography, temperature measurements, moisture measurements, bioimpedance measurements, tissue perfusion or total hemoglobin measurements, or any other technique for imaging or diagnosing tissue.

Embodiments of the present invention can also comprise methods of optimizing a monitoring system for a specific patient, e.g. by incorporating factors such as the patient's physical condition and lifestyle into a monitoring scheme. In these embodiments, a patient may be assessed for degree of neuropathy, localities of neuropathy, physical abnormalities causing pressure points, and other conditions which may be relevant to a tailored monitoring system. The number of components within a monitoring system, parameters or sensitivities of said components, directions for system use, and so forth may be determined using on one or more of these assessments. For example, a system tailored to a patient with a present but relatively low risk of injury or ulceration, e.g. a patient with a low degree of neuropathy, may comprise a periodic-monitoring device with recommendation of once-daily monitoring, whereas a system tailored to a patient with a high risk of injury or ulceration may comprise a continuous-monitoring device with recommendation of constant use as well as a periodic-monitoring device performing more complex measurements with recommendation of once or twice daily use. A system tailored to a patient with an injury or ulceration risk between these two extremes may comprise a continuous-monitoring device with recommendation of use during periods of high activity and a periodic-monitoring system with recommendation of once or twice daily use. Other combinations can be tailored to patients with these and intermediate degrees of neuropathic severity.

In one embodiment of the present invention, overall severity or degree of neuropathy may be assessed or quantified to determine an optimal monitoring system or tailor measurement types, threshold values for alert, or other parameters to a patient. This severity or degree of neuropathy may be determined by measuring nerve performance, e.g. the conduction velocity and action potential amplitude of the sural nerve; a filament-tapping test wherein a monofilament or other fine point may be pressed against predetermined points on a patient's foot, and the number and location of points at which the patient could or could not feel the filament can be used to assign a neuropathic disability score (NDS); identification of physical changes in a patient's feet, including but not limited to bunions, hammer toes, clawed toes, and Charcot Joint; or any other method or combination of methods.

Other characteristics of a patient including but not limited to age, gender, weight, BMI, and other existing medical conditions may also be utilized in embodiments of the present invention. These characteristics can be obtained from written or electronic medical records (EMR), patient interview or survey, which can be completed over a wireless device or in person, or in any other manner. These characteristics can be utilized in a variety of ways. For example, in one embodiment of the present invention, a patient's statistical likelihood of ulceration can be calculated based on his or her age, gender, weight, BMI, and other existing medical conditions, in possible conjunction with his or her degree of neuropathy. Statistics regarding the relationship between age, gender, weight, BMI, or medical conditions, in possible conjunction with his or her degree of neuropathy, with ulceration or even specific sites of ulceration can be generated by, e.g. collecting information from studies, medical practitioners, patient surveys, etc., collecting data generated by monitoring systems of the present invention, or similar methods. This type of data may be shared on a network, such as a wireless, internet, or cloud network.

Other medical conditions which may be relevant to tailoring a monitoring system to a patient's risk of injury or ulceration include but are not limited to hypoglycemia, tachycardia, hypotension, sickle cell disease, and anemia. These and other medical conditions can independently increase a patient's risk of tissue ischemia, e.g. tissue death from lack of adequate oxygenation, such that harmful external stimuli, such as excessive amounts of prolonged pressure application, may kill or damage tissue more quickly in a patient with one of these other conditions than in a patient without.

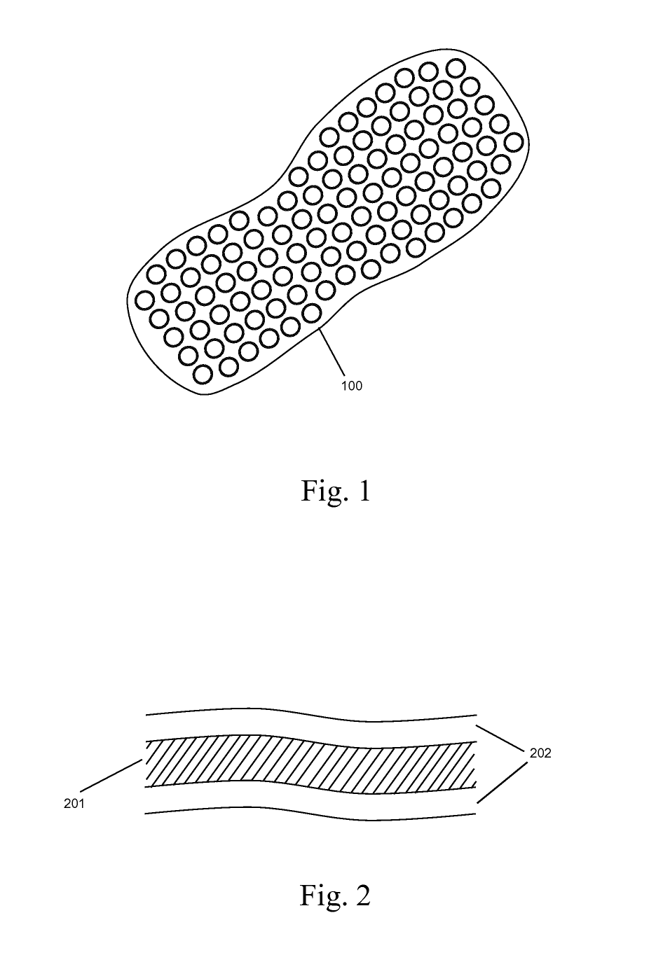

In one embodiment of the present invention, a continuous-monitoring device may be in the form of an insole. The insole can measure the amount of pressure being applied to a plurality of locations on the bottom of a foot. FIG. 1 is a diagram showing an exemplary pressure-sensing insole of one embodiment of the present invention. Insole 100 may comprise an array of sensors including but not limited to capacitive sensors, piezoelectric sensors, electrical impedance tomographic (EIT) sensors, or resistive sensors. An array may comprise between 5 and 50 sensors, 50 and 100 sensors, 100 and 150 sensors, or more. An array may further comprise between 60 and 130 sensors, 70 and 120 sensors, 80 and 110 sensors, or 95 and 105 sensors, inclusive. In other embodiments of the present invention, an array may have greater than 100 sensors, for example between 100 and 200 sensors, 200 and 300 sensors, 300 and 400 sensors, or 400 and 500 sensors.

Sensors may have any shape, including but not limited to circular, square, elliptical, rectangular, or otherwise polygonal. The surface area of individual sensors may be between 0 cm² and 20 cm², inclusive, and including but not limited to between 0 cm² and 15 cm², 0 cm² and 10 cm², 0 cm² and 5 cm², 0 cm² and 4 cm², 0 cm² and 3 cm², 0 cm² and 2 cm², 0 cm² and 1 cm² , 0 cm² and 0.5 cm², and 0 cm² and 0.25 cm², or any integer or non-integer area between the enumerated values. Sensors may be positioned with negligible separation between adjacent sensors or may be separated by a predetermined distance. A distance between adjacent sensors of an array may be up to 1 cm, 9 mm, 8 mm, 7 mm, 6 mm, 5 mm, 4 mm, 3 mm, 2 mm, or 1 mm, inclusive, or any integer or non-integer length between the enumerated values.

In one embodiment of the present invention, insole 100 can comprise an array of piezoelectric sensors. Piezoelectric materials, e.g. crystals, some ceramics, or biological matter, can accumulate charge in response to applied mechanical stress. Piezoelectric sensors may not require power to generate a signal, allowing insole 100 in this embodiment to require relatively little power. This embodiment may be particularly suited for patients who demonstrate a low degree of neuropathy or who may require continuous monitoring only during strenuous activity, e.g. prolonged walking; piezoelectric sensors can detect changing but not static pressure.

In another embodiment of the present invention, insole 100 can comprise an array of piezoresistive sensors. Piezoresistive sensors can measure changes in the resistivity of a semiconductor due to applied mechanical stress. Sensors in this embodiment may comprise micro-machined silicon diaphragms with piezoresistive strain gauges fused to silicon or glass backplates, or other configurations. Such sensors may be particularly low-cost to manufacture, can detect both static and varying pressures, and can be highly resistant to many types of noise. A temperature sensor or sensors may be included in this embodiment, and a predetermined correlation between temperature and semiconductor resistivity can be applied during signal processing to account for effects from spatial and temporal temperature variations.

In another embodiment of the present invention, insole 100 can comprise an array of capacitive sensors. Capacitive sensors may comprise variable capacitors, the capacitances of which can be related to the thickness of a dielectric material between two conductive plates, e.g. the distance between the plates. Compression of a variable capacitor by an external load can decrease this thickness such that changes in capacitance can be correlated with applied pressures or loads.

FIG. 2 is a diagram showing a cross-section of one embodiment comprising an array of capacitive sensors. Layers 202 may be fabricated from a conductive material. Conductive materials that may be utilized include but are not limited to metals, e.g. silver, copper, aluminum, or other conductive metals; conductive polymers, e.g. intrinsically conductive polymers (ICP); graphene; and combinations or alloys of these or other conductive materials. In one embodiment of the present invention, layers 202 can be made from a conductive fabric. Conductive fabric may comprise thread of a fabric material such as nylon, polyester, or similar, which has been coated or plated with a conductive metallic material including but not limited to silver, cobalt, nickel, copper, and combinations thereof. Layers 202 may be each be between 50 μm and 0.2 mm thick, inclusive. The thickness of each of layers 202 may further be between 60 μm and 0.18 mm, 70 μm and 0.16 mm, 80 μm and 0.14 mm, or 90 μm and 0.12 mm, inclusive, and any thickness within or between the enumerated ranges.

Inner layer 201 may be fabricated from a dielectric material. Dielectric materials which may be utilized include but are not limited to silicon, rubber, polyester, polyimide, titanium dioxide, aramid, plastic dielectrics, other polymers and similar materials. The thickness of an uncompressed inner dielectric layer may be between 1 μm and 1 mm, inclusive. For example, a dielectric layer may have a thickness between 1 μm and 0.1 mm, 0.1 mm and 0.2 mm, 0.2 mm and 0.3 mm, 0.3 mm and 0.4 mm, 0.4 mm and 0.5 mm, 0.5 mm and 0.6 mm, 0.6 mm and 0.7 mm, 0.7 mm and 0.8 mm, 0.8 mm and 0.9 mm, or 0 9 mm and 1 mm, inclusive, and any thickness within or between the enumerated ranges. In one embodiment of the present invention, an inner dielectric layer can have an uncompressed thickness between 0.4 mm and 0.6 mm.

Inner dielectric layer 201 may be compressible by at least 25% and up to 75%. Pressures applied to a sensor of this embodiment may be determined according to the capacitance equation C=KE₀A/D where C represents capacitance, K the dielectric constant of the inner layer, E₀ is an electric constant, and D is the distance between layers 202, e.g. thickness of inner layer 201.

Other embodiments of the present invention may comprise additional, alternating conductive and dielectric layers to increase the overall capacitance of the sensor. The materials of these alternating layers can be the same for each conductive layer or for each dielectric layer, or may comprise a plurality of materials. A total of 5, 7, 9, 11 or more layers may be utilized.

Sensors in embodiments of the present invention can be configured to measure pressures in the range of 0 to 4 MPa. Sensors can also be configured to measure pressures between 0 and 100 psi, 0 and 80 psi, 0 and 60 psi, or 0 and 40 psi, inclusive, or any other pressures within or between the enumerated ranges.

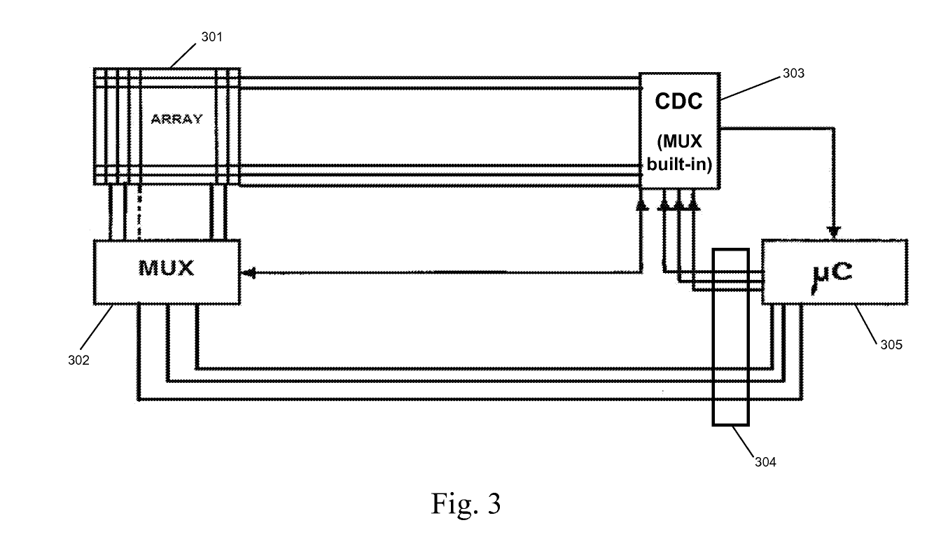

Capacitive sensor elements of a pressure-sensing array of embodiments of the present invention can be individual sensors arranged in perpendicular rows and columns, in offset rows, in a space-efficient manner to cover the insole, or in any other manner. Alternatively, a pressure-sensing array may comprise strips of conducting material organized into a plane of rows and a plane of columns, the two planes being separated by a dielectric and thereby forming capacitive elements where said rows and columns overlap. Other sensor configurations may also be utilized. A pressure-sensing insole or similar embodiments of the present invention may comprise a single capacitive array but may also comprise multiple arrays. Multiple arrays can cover the entire insole or may be arranged for selective coverage. Arrays may be defined by sets of capacitors sharing a single control or processing circuit, as described below in FIG. 3, or in any other manner.

Sensor arrays of embodiments of the present invention may further be laminated, enclosed by protective layers, or coated with a protective layer against shear stress, friction effects, moisture, or other factors that could damage sensors or bias sensor signals. Laminates or protective layers can include without limitation silicon, polyeurethane, rubber, foam, and polyimide.

FIG. 3 is a diagram showing a processing or control circuit coupled to an array of capacitive sensors of one embodiment of the present invention. The circuit of this embodiment comprises a capacitive array 301, a microcontroller 305, a multiplexer 302, a capacitance-to-digital converter 303 with an associated multiplexer, and optionally counter 304.

Microcontroller 305 may be a low-power or ultra-low-power microcontroller, e.g. a microcontroller which consumes less than 10 mA in an active mode. Microcontroller may be an 8-bit, 16-bit, 32-bit, or other size microcontroller. CDC 303 may be a capacitance-to-digital converter with a number of channels tailored to the number of elements in array 301 or another system parameter. It may also be an 8-bit, 16-bit, 32-bit, or other converter. CDC 303 may be a low-power or ultra-low-power device. CDC 303 may have a built-in multiplexer or demultiplexer. Alternatively, output from CDC converter may be coupled to an external multiplexer.

Multiplexer 302 may be an analog multiplexer or chain of multiplexers. It may have the same number of channels as CDC 303 or a different number of channels. For example, multiplexer may have between 2 and 32 channels, inclusive, or greater than 32 channels. Elements along columns of array 301, e.g. capacitive plates of variable capacitors aligned in one direction of the array, may be connected to one another and may be connected to an input channel of multiplexer 302. Rows and columns of array 301 may or may not be perpendicular to one another. Multiplexer 302 can also be coupled to counter 304. Counter 304 can be controlled by microcontroller 305 and can select a given channel of multiplexer 302, e.g. a column of the array. Selecting a column of array 301 can establish a connection between said column and microcontroller 305. For example, selecting a column can apply a predetermined voltage to the column, said predetermined voltage being set by microcontroller 305. In one embodiment of the present invention, said predetermined voltage can be 0 V or ground. When a column is not selected, its voltage can be floating or may be isolated from other components of the control circuit.

Elements along a row of array 301 can be coupled to one another and to a channel of CDC 303. Counter 304, coupled to microcontroller 305 and to a multiplexer within or associated with CDC 303, can select a given row of array 301 to be read by CDC 303. CDC 303 may determine the capacitance of elements connected to one of its channels by applying an AC signal to the channel and performing an impedance measurement. If a single column of array 301 is selected, e.g. grounded, and other columns are floating, then application of an AC signal along a row of array 301 may only generate a significant capacitive coupling at a sensor with a grounded plate, e.g. the sensor falling in the column selected by multiplexer 302 and row selected by CDC 303. Impedance measured by CDC 303 will therefore be attributable to capacitance of the selected sensor. CDC 303 can convert a capacitance value into a digital signal, which it can send to microcontroller 305.

In one embodiment of the present invention, sensor elements of array 301 can be read row by row. In this embodiment, multiplexer 302 may cycle through each column of array 301 while a single row is activated by the multiplexer associated with CDC 303. Once each column has been selected, the new row can be activated within which columns can be cycled, and so forth. In an alternative embodiment of the present invention, elements can be read column by column. In this embodiment, multiplexer 302 may hold a given channel of array 301 selected, e.g. grounded, while a multiplexer associated with CDC 303 cycles through rows of array 301.

In another embodiment of the present invention, the pattern in which capacitive sensors are read can be tailored to at-risk regions of a patient's foot. At-risk regions can be determined in real-time, for example from data provided by previous reads of the array or from an additional type of sensors, or may be determined by one of the previously described evaluative procedures. For example, the sensors below at-risk regions on the sole of a patient's foot may be read more frequently than sensors below relatively low-risk regions. The recording of a pressure above a predetermined threshold at a sensor or subset of sensors may trigger the pattern to readjust and read said sensor or subset more frequently than other sensors of the array. Sensors neighboring a sensor or sensors exceeding the threshold pressure in a given read can also be included in a subsequently tailored read pattern to account for the possibility of relative movement between a patient foot and the insole.

Other patterns or combinations of the patterns described above can also be utilized. Multiplexer 302 and a multiplexer associated with CDC 303 can read and write array positions or channels as least-significant bit (LSB) values, most-significant bit (MSB) values, or both. For example, in one embodiment of the present invention, one multiplexer reads MSB and the other LSB, such that the first half of a number of bits in a byte designate a row in the array and the second half of the bits designate a column, or vice versa. Counter 304 can cycle through possible configurations of half the bits, e.g. LSB values, before changing the value of bits in the other half, e.g. MSB value. In this manner, counter 304 can cycle through rows and columns of the array. Alternatively, microcontroller 305 can express a specific array location designated by a single byte directly to both multiplexers. Sampling frequency of each sensor may be less than 1 Hz, 1 Hz, 2 Hz, 3 Hz, or higher, or any frequency within or between the enumerated values. The frequency at which a sensor is read, e.g. at which the pressure applied at a specific site on a patient's foot is sampled, can be increased by lessening the number of sensors being read by a CDC or other component with a given throughput rate.

Microcontroller 305 can assign digitized capacitance values for sensors of array 301 to a memory array. Microcontroller 305 may further process data received from CDC 303, may send data to an external receiving device or processor, such as a hybrid hard drive, computer, computer network, computing network, or other device or network with processing or computing capabilities.

Interconnects within the circuit of FIG. 3 and similar embodiments of the present invention may be configured to accommodate an amount of flexing, bending, or other motion. Interconnect materials can include without limitation zero-insertion-force (ZIF) connectors, conductive epoxy, polyimide film, and other polymer films or adhesives.

These and other embodiments of the present invention can be powered by any one of a variety of external or integrated power supplies, including but not limited to batteries such as air-zinc, solid-state, coin cell, and lithium ion batteries, capacitive storage devices, or energy-harvest systems. The level of integration of a power source with a continuous-monitoring device can be complete, e.g. embedded or incorporated within the device; partial, e.g. incorporated or closely attached but possibly removable or replaceable; or external, e.g. relatively free from the device. An external power source may be carried in or incorporated into another article the patient wears. For example, a power supply for a pressure-sensing insole may be positioned in or on a shoe. A processing or control circuit, e.g. of FIG. 3, can utilize low-power components as described, which may allow for a relatively small or completely incorporated power supply.

In one embodiment of the present invention, a power source comprises a kinetic energy harvest system. The kinetic energy harvest system of this embodiment can include electroactive polymers or piezoelectric ceramics positioned beneath or within a heel area of an insole or shoe. For example, a layer or layers of a dielectric elastomer, a type of electroactive polymer which produces electricity upon compression or deformation, can be incorporated in a manner such that patient motion will compress the layers, e.g. with each patient step. An exemplary system which may be utilized has been described by Kornbluh, Eckerle and McCoy. Other kinetic energy harvest systems which may be utilized in embodiments of the present invention may comprise vibration-sensitive varactors, e.g. variable capacitors, which can convert mechanical vibrations into electricity, or other mechanisms of mechanical energy conversion.

In another embodiment of the present invention a power source may comprise a thermal energy harvest system. For example, a thermoelectric generator (TEG) may be utilized; a temperature gradient may be maintained between two conductors to generate a voltage difference and electric current. The temperature gradient may be maintained by positioning one conductor of the TEG in contact with or near the wearer's skin, a region of high friction in a shoe or insole, or any other relatively warm region, while thermally isolating or otherwise minimizing the temperature of the second conductor. The voltage difference available from a TEG in this embodiment of the present invention may be approximately determined by V=(S₂−S₁)·(T₂−T₁) where V is the voltage generated, S₂ and S₁ are the thermopowers, e.g. Seebeck coefficients, of the warmer conductive element and cooler conductive element respectively, and T₂ and T₁ are the temperatures of these two elements, respectively. A TEG may be fabricated and tailored to a continuous monitoring device of the present invention, or a commercially available TEG may be utilized.

In another embodiment of the present invention, a sensor array may be configured to monitor less than a full insole. Patient-specific pressure points or statistically likely sites of ulcer development may be monitored. As previously described, locations of physical abnormalities such as bunions, hammer toes, clawed toes, Charcot Joint, or others may be recorded during a consultation with a patient. Sensors or sensor arrays may be placed on insole or shoe locations corresponding to locations of these abnormalities. A sensor configuration may also sense pressure at the point of an insole where ulcers develop most commonly, e.g. under the toes, metatarsals, and heels.

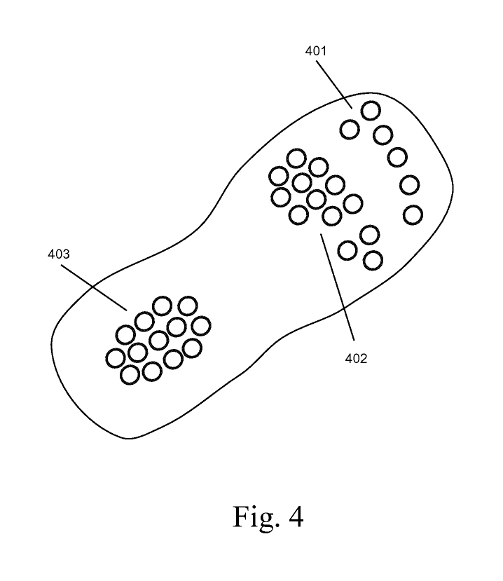

FIG. 4 is a diagram showing an insole sensor configuration of one embodiment of the present invention for monitoring statistically likely sites of ulcer development. In the embodiment of FIG. 4, sensors can be positioned under one or more toe positions 401, metatarsal positions 402, and heel positions 403.

Embodiments of the present invention that have been described may be incorporated in an orthotic, shoe, or other structure. For example, an insole may form an uppermost, inner, or lowermost layer of a custom orthotic. Alternatively, a means of affixing an insole to the top or bottom of an off-the-shelf orthotic may be provided. In one embodiment of the present invention, a custom orthotic can be designed to accommodate or alleviate pressure around one or more previously described abnormalities. Pressure alleviation may be accomplished by varying the surface contours of or material composition across a custom orthotic or in some other manner.

In another embodiment of the present invention, a continuous-monitoring device can comprise a sandal, flip-flop, slipper, or similar article of footwear. One or more pressure-sensing arrays that have been described may be incorporated in the sole of said footwear, for example as a top layer in contact with the patient foot, as a mid-layer between an upper layer and lower protective layer, or in any other manner. An upper layer may be orthotic-like or provide light cushioning. Sensors may also be positioned on other parts of the footwear, such as a band over the top of the foot, between the toes, or other points of skin contact. One or more perfusion sensors, oxygenation sensors, e.g. total hemoglobin sensors, temperature sensors, or other sensors can also be included in the sole or other parts of the footwear. This embodiment may be designed for use with our without a sock. In other embodiments of the present invention, a continuous-monitoring device can surround the foot, such as in the form of a sock or shoe. Pressure sensors and other sensors can cover the inner surface of the shoe or sock or may be located at specific sites, such as sites with a predetermined risk of injury, joints, or other prominences. Such sites can be identified by preliminary evaluation as previously described or in any other manner.

Data acquired during each read of sensors or sensor arrays in embodiments of the present invention can be processed in a variety of manners. In some embodiments of the present invention, sensor data can be translated into or recorded as a count or counts, which can be assigned to a bin representing a particular sensor or subset of sensors. A translation of sensor data, e.g. pressure values, into binned counts may comprise assigning a count to a sensor bin if the pressure registered by that sensor in a given read exceeds a predetermined threshold value. A translation of sensor data into binned counts may also comprise scaling the number of counts assigned to a bin as a function of rate and load, e.g. of psi/sec or similar ratio values.

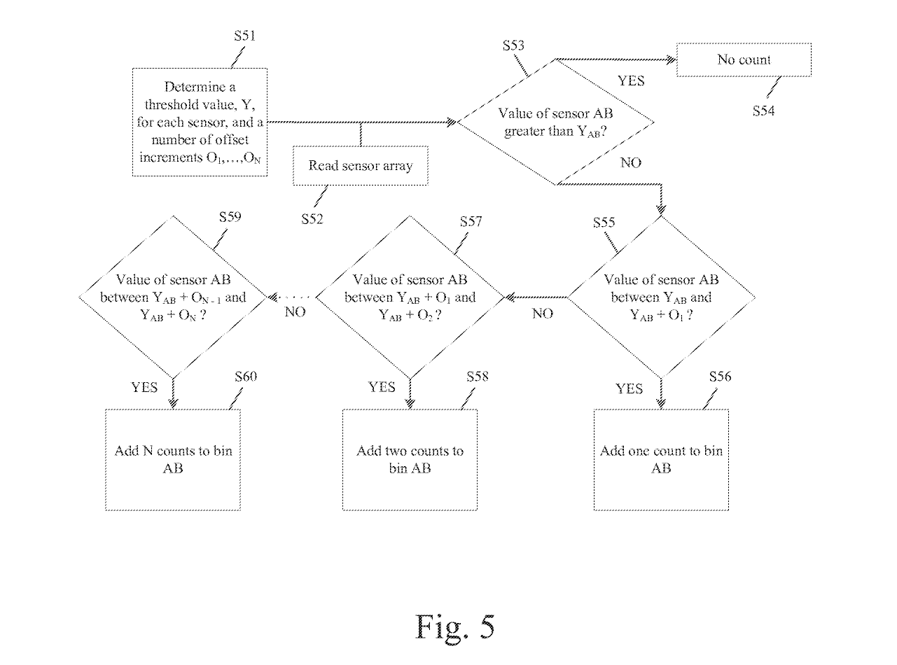

FIG. 5 is a flow diagram representing a method of data-binning of one embodiment of the present invention. A first step S51 in the method of FIG. 5 can be determining a suitable threshold pressure for each sensor, e.g. Y_{AB}, where Y can be a threshold value and A and B can be identifying indices for the sensor, e.g. a row number and column number. In one embodiment of the present invention, a threshold value can be constant across all sensors in an array or configuration. Selection of a suitable threshold value may be patient-independent, e.g. a value considered potentially harmful for most patients, or patient-specific, e.g. a value determined with consideration to a given patient's degree of neuropathy, weight, tissue perfusion, tissue oxygen saturation, physical abnormalities, or other characteristics. In pressure-sensing embodiments of the present invention, a patient-independent threshold value may be an integer or non-integer value between 10 psi/sec and 50 psi/sec, inclusive. A patient-independent threshold value may further be an integer or non-integer value between 15 psi/sec and 45 psi/sec, 20 psi/sec and 40 psi/sec, 25 psi/sec and 35 psi/sec, or 27 psi/sec and 33 psi/sec, inclusive. In some embodiments of the present invention, a threshold value may be less than 10 psi/sec, such as when a specific site or wound is being monitored.

In additional embodiments of the present invention, pressure threshold values can be adjusted based on data from another continuous or periodic-monitoring device; a patient's medical record or electronic medical record; data aggregated from many patients using a device or similar device, e.g. over a cloud or other physical or wireless network; instruction from a medical provider; or any similar source. For example, embodiments of the present invention described below can be utilized to determine a relationship between pressure-loading and nonblanchable erythema or tissue ischemia, e.g. a relationship between an amount of pressure load, time of pressure load, and onset of nonblanchable erythema or ischemia. This relationship or set of relationships can be utilized to set an appropriate pressure threshold or pressure thresholds.

Updated or new pressure threshold information can be transmitted to a pressure-sensing continuous-monitoring device. Pressure thresholds may be updated in real-time, e.g. while the device is in use.

Step S51 can also comprise determining or selecting values for a number of offset increments from pressure thresholds, e.g. incremental values exceeding the first pressure threshold value Y_{AB}. A set of N offset increments or ranges, e.g. O₁, O₂, . . . , O_{N}, may be determined and utilized for all sensors in an array. The set can be based on values considered to represent increasing levels of injury risk for most patients, or can be tailored to specific patient conditions, e.g. degree of neuropathy or pre-existing wounds. In an alternative embodiment of the present invention, a set of N offset increments or ranges can be determined for each sensor or for a subset of sensors.

Offset increments in a set can be of equal or varying size. For example, offset values may be configured such that O_{N}=N*c*O₁ where c is a constant or that a value of O_{N} is relatively independent of the value of O₁. In pressure-sensing embodiments of the present invention utilizing offset increments of equal size, O₁ may be any integer or non-integer value between 0 psi/sec and 10 psi/sec inclusive. O₁ may further be between 0 psi/sec and 5 psi/sec, 0 psi/sec and 4 psi/sec, 1 psi/sec and 5 psi/sec, or 1 psi/sec and 4 psi/sec, inclusive, or any integer or non-integer number of psi/sec within or above the enumerated ranges. The constant, c, may be 0.25, 0.5, 0.75, 1, 2, 3, 4, 5, or any other integer or non-integer value between the enumerated values. Any number of increments may be utilized in a set, though the number of increments may be related to the processing time required to count and bin sensor data from each read.

In step S52 sensors or a sensor array can be read, and the value of each sensor, e.g. sensor AB, compared to a respective threshold value, e.g. Y_{AB}, as shown in step S53. If said value is less than Y_{AB}, no counts may be added to a bin corresponding to sensor AB, e.g. bin AB, as in step S54. If said value is greater than or equal to Y_{AB}, a count or counts may be assigned to a bin. If offsets have been determined in step S51, the amount by which a pressure reading exceeds threshold Y_{AB} can be translated into a predetermined number of counts. For example, as shown in FIG. 5, in step S55 a pressure reading having exceeded Y_{AB} can be compared to Y_{AB}+O₁, where O₁ is a first offset value. If a pressure reading associated with sensor AB is greater than threshold Y_{AB} but less than Y_{AB}+O₁, a first predetermined number of counts may be added to bin AB. As shown in step S56 this first predetermined number of counts may be one count.

If a pressure reading is greater than Y_{AB}+O₁ and further offset values have been determined, it may be compared to Y_{AB}+O₂, where O₂ is a second offset increment, as shown in step S57. A second predetermined number of counts, e.g. two counts, may be added to bin AB as shown in step S58 if the pressure reading is between Y_{AB}+O₁ and Y_{AB}+O₂. If N offsets have been determined, this process may be repeated up to a comparison of the pressure reading against Y_{AB}+O_{N} as shown in step S59. If the reading is greater than Y_{AB} +O_{N-1} but less than Y_{AB}+O_{N}, a predetermined number of counts, e.g. N counts, may be added to bin AB in step S60. The process described by steps S53 through S59 or some subset thereof may be repeated for each sensor of the sensor array read in step S52.

In another embodiment of the present invention, data can be binned according to subsets of sensors in an array. For example, n/m subsets can be created where n represents the number of sensors in an array and m represents the number of sensors in each subset. Alternatively, subsets differing in size, e.g. number of sensors, can be created; for example, sensors associated with a particular region of tissue, e.g. a metatarsal or toe, can be grouped as a subset. Subset boundaries may be predetermined or may be dynamic, e.g. determined during processing for each read of the array; patterns in pressure loading can be recognized to delineate toes, metatarsals, a heel, and so forth.

In one such embodiment, bin counts may be assigned according to two threshold values. For example, one threshold value can be a sensor value, e.g. Y from the embodiment of FIG. 5, and another threshold value can be a number of sensors within a subset that must exceed the sensor threshold value for a count to be recorded in the subset bin, e.g. p of m sensors in a subset must exceed a threshold value for a count to be recorded in the subset bin.

Tissue perfusion, oxygen saturation, total hemoglobin, or other similar metrics can be related the likelihood of tissue injury or be indicative of injury or ulcer onset. In one embodiment of the present invention, one or more of these values can be measured by a continuous or periodic monitoring device, taken from a patient's medical record, e.g. an electronic medical record, or otherwise acquired. These metrics can be utilized independently to monitor tissue health or in conjunction with other metrics, e.g. with pressure data acquired by embodiments described above.

In one embodiment of the present invention, a continuous monitoring device can comprise a system for blanch testing, e.g. in the form of an insole, sock, sock-liner, slipper, patch, shoe, or similar article of footwear. A blanch test may identify lowered tissue perfusion or the presence of nonblanchable erythema. Nonblanchable erythema, e.g. tissue redness which does not reduce upon pressure application, can indicate a reversible, early-stage pressure ulcer. Tissue redness can indicate an increased supply of blood to the region and may therefore be identified by total hemoglobin measurement, hemoglobin being a component of blood. A light source and sensor can be configured to measure total hemoglobin, tissue color, or any similar metric at one or a plurality of positions on the sole of a patient's foot. One or more pressure sensors, pedometers, or other indicators can be utilized to trigger measurement said tissue metric once or more while pressure is applied to the tissue, e.g. while a patient's foot is on the ground, and once or more while little or no pressure is applied to the tissue, e.g. while a patient's foot is raised between steps. A difference between measurement results with and without pressure applied can be quantified or otherwise analyzed and related to the amount and rate of blood supply to the tissue region. A source and sensor may be positioned on a portion of the device corresponding to a load-bearing region of the foot, e.g. the ball or heel of the foot, to maximize the difference in pressure between the two or more measurements.

A total hemoglobin measurement configuration may comprise one or more light sources and one or more light sensors. A source and sensor may be positioned on or in the footwear, e.g. flush with an upper surface contacting tissue of the bottom of a patient's foot, and separated from one another by a distance between 50 μm and 1.5 cm, inclusive. A source and sensor configured for total hemoglobin measurement of underlying tissue may further be separated by a distance of 200 μm to 6 mm, 5 mm to 1.2 cm, 6 mm to 1.1 cm, and 7 mm to 1 cm, inclusive, and any other integer or non-integer distance within the enumerated ranges. A source or sources may emit light of one, two, three, or more wavelengths. In one embodiment, a source or plurality of sources may emit light with a wavelength or wavelengths between 500 nm and 700 nm, 700 nm and 1400 nm, 800 nm and 900 nm, or 800 nm and 820 nm, inclusive, and any other wavelengths within or between the enumerated ranges. A wavelength matching an isosbestic point of two or more types of hemoglobin, e.g. oxyhemoglobin and deoxyhemoglobin, may be utilized. Visible light and near-infrared light may be utilized. Sources can include without limitation light-emitting diodes (LED's), LED chips, laser diodes, and optical fibers. In one such embodiment, sources can comprise LED chips or similar sources with diameters between 150 μm and 400 μm, 160 μm and 350 μm, 170 μm and 300 μm, 190 μm and 250 μm, or 200 μm and 240 μm, inclusive, or any other integer or non-integer number of micrometers within the enumerated ranges. In some embodiments, sources can also be larger, e.g. greater than 400 μm in diameter.

In another embodiment of the present invention, a continuous monitoring device can comprise a tissue oxygenation measurement system configured for measurements on the soles of feet. Poor or relatively low, e.g. relative to other parts of the body, oxygen saturation in a region of tissue can be indicative of tissue ischemia. Prolonged ischemia can result in tissue necrosis, e.g. death, one mechanism by which diabetic foot ulcers form. Oxygenation or oxygen saturation may be measured by emitting two or more different wavelengths into tissue, measuring the absorption of each, and determining the ratio of oxyhemoglobin to deoxyhemoglobin in underlying blood based on predetermined absorption characteristics of the hemoglobin types. In this embodiment of the present invention, a similar array of light-emitting sources and light sensors can be utilized to measure blood tissue oxygen saturation as described for a total hemoglobin measurement system. However, sources may emit at least two wavelengths of light in the visible or near-infrared range, e.g. between 400 nm and 1400 nm.

Measurements may be acquired at any regular or irregular predetermined or triggered time intervals. In one embodiment, measurements may be continually acquired during periods of device use, at any predetermined time interval including but not limited to between 1 ms and 60 s, inclusive. Time intervals between measurements at a given site can include without limitation 1 ms to 300 ms, 150 ms to 450 ms, 450 ms to 1 s, 1 s to 30 s, and 30 s to 60 s, and any integer or non-integer number of seconds or milliseconds within the enumerated ranges. In some embodiments, this interval may be greater than 1, 5, 10, or 30 minutes.

Embodiments of the present invention comprising one or more light sources and sensors may be implemented in articles of footwear that contact the tissue of a foot directly. Various articles of footwear can meet this condition or be configured to meet this condition. For example, in one embodiment of the present invention the light sources and sensors can be incorporated in a sock or sock-liner, e.g. a relatively thin sock-like article worn beneath a regular sock to reduce friction or irritation. In another embodiment, sources and sensors can be incorporated in a slipper, e.g. for continuous monitoring while a patient is at home. In another embodiment, sources and sensors can be incorporated in a flip-flop or sandal. Sources and sensors can also be incorporated in a closed shoe, which can be configured for wear without a sock. This configuration may comprise use of lining materials that are low-friction, slipper-like, cushioning, odor-resistant, or having similar properties to enable comfortable and non-damaging usage without a sock.

In another embodiment, sources and sensors can be incorporated in a patch that can remain in contact with a foot or specific region of a foot. For example, the patch may be tailored to monitor a specific region such as a load-bearing region, e.g. heel or ball of the foot; vulnerable region, e.g. physical deformity or recovering wound; or any other region. The patch may alternatively be relatively large, e.g. similar to a thin, flexible insole that can be worn inside of a sock. A patch may be held in contact with tissue by insertion into a sock, slipper, or similar, or may be coated with an adhesive to stick to tissue upon application. The patch may be made from one or more layers of any flexible material including but not limited to fabric, rubber, plastic, latex, another polymer, or any combination thereof

Embodiments of the present invention can also comprise continuous monitoring devices that combine any of the above tissue monitoring techniques, e.g. pressure sensing, blanch-testing, total hemoglobin measuring, or oxygen saturation measuring. For example, one embodiment of the present invention can comprise an insole, sock, sock-liner, slipper, patch, shoe, or similar article of footwear with an embedded pressure sensing array and total hemoglobin measurement array. Alternatively, this embodiment can comprise two articles of footwear, e.g. one with pressure-sensing capabilities and the other with blanch-test capabilities. In this embodiment, pressure data can be utilized to trigger a blanch test, e.g. while the patient is walking or there is a regular application and removal of pressure to the foot. In one embodiment, pressure data can be analyzed to determine a first level of at-risk regions in a patient's foot, e.g. that are experiencing high pressure loads, and hemoglobin data can indicate a second level of at-risk regions in the foot, e.g. that are demonstrating nonblanchable erythema. In another embodiment, pressure data and hemoglobin data can be analyzed in conjunction with one another following a predetermined time period to build a predictive relationship between a pressure loading amount and time that can result in nonblanchable erythema. This data or a relationship derived therefrom, e.g. through analytical methods, data-fitting methods, computer learning methods, or similar, can be stored in a patient's medical record, electronic medical record, device memory, or any other location.

In another embodiment of the present invention, pressure data from a continuous monitoring device can be used to trigger tissue oxygenation measurements by the same or a second continuous monitoring device. For example, detection of any amount of pressure loading, e.g. above a predetermined pressure threshold or time threshold, can trigger one or more tissue oxygenation measurements to be taken in the pressure-loaded regions. Pressure data and oxygenation measurement data can also be analyzed in conjunction with one another following a predetermined time period to build a predictive relationship between pressure loading amount and time that can result in tissue ischemia in a given patient, e.g. through analytical methods, data-fitting methods, computer learning methods, or similar.

In another embodiment of the present invention, blood pressure measurements may be utilized as another metric of tissue health. Blood pressure can be related to or indicative of high or low tissue perfusion levels. Blood pressure can be measured by an independent periodic monitoring device, e.g. an in-home or automated pressure cuff, or continuous monitoring device. This data can also be analyzed in conjunction with data acquired from any of the continuous monitoring devices that have been described, or may affect the measurement rates or alarm thresholds for, e.g., a pressure sensing insole, as tissue injury can be more likely when perfusion is low.

Data processing, such as the binning and counting described by FIG. 5 or similar methods, can be done by a microcontroller, an external receiving and processing device, a physical or wireless, e.g. cloud, computing network, or any similar system. In one embodiment of the present invention, an amount of processing, e.g. evaluation of each sensor against a threshold value, may be done by a microcontroller while further processing, e.g. analysis or dynamic mapping of resultant data, can be carried out by an external receiving device or network.

Data or signals from continuous-monitoring devices of the present invention can be transmitted to external receiving or processing devices or networks for processing, analysis, display, storage, or other applications. Communication protocol between a continuous monitoring device and an external receiving device can include without limitation Bluetooth, ISM band, near-field, WiFi, body network, ANT+, and similar types of communication protocols. External devices which may be utilized include but are not limited to mobile phones, watches, wrist bands, desktop and laptop computers, electronic tablets, and any type of processing unit or display screen.

Data can be transmitted regularly while a continuous-monitoring device is worn by a patient, or in limited bursts or periods based on connectivity strength, patient activity, or other factors. In one embodiment of the present invention a communication rate can scale according to the activity of a wearer. For example, the rate of communication can be increased when the pressure-loading sensed by an insole or shoe in embodiments of the present invention exceeds a predetermined value that can be considered indicative of walking or other weight-bearing activity. A sensor array may be read at predetermined intervals, and an internal processing device can increase communication to an external receiving device or network only once a significant amount of pressure or activity is sensed. Alternatively, a single sensor or subset of sensors can be read to monitor for weight-bearing activity, in which case the full array of sensors may be activated along with a communication-rate increase once activity above said threshold is detected.

Embodiments of the present invention can further comprise methods of communicating or displaying pressure data to a patient or medical practitioner. In one embodiment of the present invention, an external receiving device or processor can generate a dynamic pressure map of the sole of a patient's foot in real time. For example, a monitoring system can comprise an application for a smart phone or other mobile processing device that receives sensor data and generates a color map or 3D map representing the current application of pressure across the sole of the foot.

In another embodiment of the present invention, an external receiving device or network can aggregate sensor data over predetermined time intervals and provide analyses or alerts to the patient or a medical practitioner. The external device may aggregate data from a day, a period of activity, or other longer or shorter time interval. Pressures registered at a sensor for each read of the array can be stored, averaged, or otherwise aggregated. Similar processing as described for the embodiment of FIG. 5 can be performed on such aggregated data by the external device. A map of the sole of a foot can be generated wherein colors or third-dimension heights are assigned to regions of the sole exceeding a threshold by varying amounts. Alerts may be sent to the patient or practitioner if aggregated pressures to any site on the foot exceed a threshold amount. As described for the embodiment of FIG. 5, threshold amounts can be tailored to the region of the foot, tissue perfusion, and other patient-specific factors.

For data aggregation and other analysis purposes, individual sensors or subsets of sensors may be associated with specific sites on the sole of a patient foot; pressure values can be aggregated according to the sensor location at which they were measured. Alternatively, data can be aggregated in a manner accounting for motion of the patient foot relative to a monitoring device between reads of the sensor array or sessions of use. In this embodiment of the present invention, regions of the sole of a foot can be associated with sensor data by a method or methods including but not limited to boundary matching, image convolution, pattern recognition, or other data or image processing methods. One or more reference positions, such as the middle of the heel, toes, or other features may also be identified and utilized in conjunction with predetermined metrics of the patient foot to associate sensor data with sites of the sole. A relationship between sensors of the array and regions of the foot can be generated for each read of the array, for each session of use, or at any other predetermined interval.

In a further embodiment of the present invention, aggregated data from a continuous-monitoring device can be exported to a periodic monitoring device or correlated with data from a periodic monitoring device for analysis. In one embodiment of the present invention, data acquired by a continuous or periodic monitoring device may be added, linked, or sent to an electronic medical record (EMR). Data from multiple types of measurements may optionally be aggregated before, during, or after addition to the EMR. Data may be aggregated and may be linked to the EMR by any physical or wireless means, including but not limited to a cloud computing interface or other server interface. Data aggregation may be performed between data acquired from one or multiple monitoring devices, measurements performed during visits with a medical practitioner, or any other modes of patient data collection. These data and measurements can include without limitation tissue temperature, tissue perfusion, tissue oxygen saturation, total local hemoglobin, patient weight, pulse, heart rate, respiratory rate, localized pressure loading, pressure loading patterns, degree of neuropathy, locations of known physical deformities or other conditions, history of injury or ulceration, or any other metrics or conditions related to tissue and patient health.

One or more sensor self-calibration methods may be utilized in embodiments of the present invention to maintain accurate pressure information even in the presence of deformation or degradation of sensor materials. One such method comprises analytically determining the probable deformation, creep, or drift of sensors in a pressure-sensing array of embodiments of the present invention given the load and rate of loading detected for each sensor. Other factors which may be included in this determination may include the sensor material, dimensions, temperature during loading, position of a given sensor in the array, or other system parameters.

Another method of sensor self-calibration can comprise periodically reading the sensor array or elements of the sensor array under no pressure load. This read may be initiated while the continuous-monitoring device is not in use by a patient, for example during the night, upon powering-on, at election by the patient, e.g. pressing a button, or another time. Each sensor in the array or a subset of the array may be read and normalized, adjusted, or otherwise calibrated in a manner accounting for variations from an initial state, unloaded measurement value, or other parameters.

Self-calibration may also or alternatively be performed while the device is in use. In one embodiment of the present invention, a sensor in a position experiencing relatively low cyclic loading, such as under the inner arch of a foot, may be taken as a reference sensor in a calibration method. In this embodiment, other sensors in the array can be read in an unloaded state, e.g. between steps of a patient, while the patient is sitting, or similar, and compared to the unloaded state value of the reference sensor. A difference exceeding a predetermined level between another sensor in the array and the reference sensor can trigger recalibration of said sensor. The predetermined level may be a drift of between 1%and 10%, 1% and 5%, or 2% and 5%, inclusive. For example, any sensor whose unloaded capacitance value differs by 3% or more from the unloaded capacitance value of the reference sensor may be recalibrated.

The foregoing descriptions of specific embodiments of the present invention have been presented for purposes of illustration and description. They are not intended to be exhaustive or to limit the invention to the precise forms disclosed, and many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the claims appended hereto and their equivalents.

What is claimed is:

1. A system for monitoring a human foot comprising:
an array of pressure sensors embedded in an article of footwear configured to measure pressures applied to regions of said human foot;
a low-power control circuit for reading said array of pressure sensors;
a power source incorporated in said article of footwear configured to power said array of pressure sensors and said low-power control circuit;
an external processing unit wirelessly coupled to said low-power control circuit configured to relate said pressures applied to regions of said human foot to a number of counts associated with injury risk; and
an alert system for raising an alert if said number of counts associated with injury risk exceeds a predetermined threshold.

2. The system of claim 1 wherein said external processing unit comprises a cloud computing network.

3. The system of claim 1 further comprising a wireless connection to an electronic medical record configured to transmit pressure information to said electronic medical record.

4. The system of claim 1 further comprising a light source and sensor configured for detection of nonblanchable erythema in said regions of said human foot.

5. The system of claim 1 further comprising a light source and sensor configured for detection of tissue ischemia in said regions of said human foot.

6. A system for monitoring a human foot comprising:
an article configured to be worn in contact with tissue of said human foot;
a light source embedded in said article for emitting light with a wavelength between 400 nm and 1400 nm into said tissue;
a sensor embedded in said article between 200 μm and 1 cm, inclusive, from said light source for detecting said light from said tissue; and
a processing unit coupled to said sensor for determining a tissue health-related condition from said light.

7. The system of claim 6 wherein said article is a sock.

8. The system of claim 6 wherein said article is a slipper.

9. The system of claim 6 wherein said article is a patch.

10. The system of claim 6 wherein said light source has a diameter less than 1 mm.

11. The system of claim 6 wherein said wavelength is further between 800 nm and 820 nm, inclusive.

12. The system of claim 6 wherein said light source and said sensor are embedded at points in said article configured to contact a load-bearing region of said tissue.

13. The system of claim 6 wherein said tissue health-related condition is presence of nonblanchable erythema.

14. The system of claim 6 wherein said source is configured to emit a second wavelength of light between 400 nm and 1400 nm.

15. The system of claim 14 wherein said tissue health-related condition is tissue oxygen saturation.

16. A method for determining a relationship between an amount of pressure experienced by a region of tissue and risk of ulcer development in said region of tissue comprising:
measuring said amount of pressure experienced by said region of tissue on a human patient with a wearable pressure-sensing article over a predetermined time period;
measuring a tissue hemoglobin condition in said region of tissue with a wearable hemoglobin-measuring article over said predetermined time period; and
analyzing said tissue hemoglobin condition for signs indicative of said risk of ulcer development.

17. The method of claim 16 wherein said tissue hemoglobin condition is total hemoglobin.

18. The method of claim 16 wherein said tissue hemoglobin condition is ratio of oxyhemoglobin to deoxyhemoglobin.

19. The method of claim 16 further comprising:
determining a pressure value that affects said tissue hemoglobin condition in said region of tissue;
monitoring pressure applied to said region of tissue; and
configuring an alert system to notify said human patient if said pressure applied to said region of tissue exceeds said pressure value.

20. The method of claim 19 further comprising:
transmitting said pressure value to an electronic medical record.
